(19) United States
(12) Patent Application Publication
(10) Pub. No.: US 2025/0157611 A1
(43) Pub. Date: May 15, 2025

(54) PATCH

(71) Applicant: DAIWA CAN COMPANY, Tokyo (JP)

(72) Inventors: Toshihiro Asano, Sagamihara-shi (JP); Keisuke Hashimoto, Sagamihara-shi (JP); Tomohiko Oyanagi, Tokyo (JP)

(73) Assignee: DAIWA CAN COMPANY, Tokyo (JP)

(21) Appl. No.: 19/022,918

(22) Filed: Jan. 15, 2025

Related U.S. Application Data
(63) Continuation of application No. PCT/JP2023/026126, filed on Jul. 14, 2023.

(30) Foreign Application Priority Data
Jul. 15, 2022 (JP) 2022-114246

(51) Int. Cl. G16H 20/10; A61M 35/00; G06K 19/06; H05K 9/00

(52) U.S. Cl. CPC G16H 20/10; A61M 35/10; H05K 9/0081; A61M 2205/3561; A61M 2205/6009; A61M 2205/6063; A61M 2209/06; G06K 19/06037

(57) ABSTRACT

The patch includes a support body, a plaster provided on one main surface of the support body, a communication device provided on another main surface of the support body and wirelessly communicating with a communication terminal, and an electromagnetic wave shielding member provided on at least one of the plaster and the communication device and allowing the communication device to be in an uncommunicable state.

### CROSS-REFERENCE TO RELATED APPLICATION

This application is a Continuation Application of PCT Application No. PCT/JP2023/026126, filed Jul. 14, 2023 and based upon and claiming the benefit of priority from Japanese Patent Application No. 2022-114246, filed Jul. 15, 2022, the entire contents of all of which are incorporated herein by reference.

### FIELD

The present invention relates to a patch for use in a system that supports use of a medicine.

### BACKGROUND

Conventionally, there has been adopted a means for instructing the use of medicine prescribed from a medical institution or a dispensing pharmacy according to a medicine bag or an instruction sheet describing a medication use method so that a patient does not use an incorrect medicine.

In addition, Jpn. PCT National Publication No. 2016-523159 discloses a means for managing and notifying a patient's medication use status by using a wireless communication device in order to avoid a patient's forgetting to use medicine. Jpn. PCT National Publication No. 2016-523159 discloses a technique of providing an electronic component in a patch to be applied to a patient's skin.

### SUMMARY

According to one aspect of the present invention, a patch includes a support body, a plaster provided on one main surface of the support body, a communication device provided on another main surface of the support body and wirelessly communicating with a communication terminal, and an electromagnetic wave shielding member provided on at least one of the plaster and the communication device and allowing the communication device to be in an uncommunicable state.

### BRIEF DESCRIPTION OF THE DRAWINGS

FIG. 1 is an explanatory diagram illustrating an example of a configuration of a medication use support system using a patch according to an embodiment of the present invention.

FIG. 2 is an explanatory diagram illustrating an example of a configuration of the medication use support system.

FIG. 3 is a plan view illustrating a configuration of the patch.

FIG. 4 is a cross-sectional view illustrating a configuration of the patch.

FIG. 5 is a bottom view illustrating a configuration of the patch.

FIG. 6 is a plan view illustrating a state in which the patch is placed in a package.

FIG. 7 is an explanatory view illustrating a state in which the package is opened.

FIG. 8 is an explanatory view illustrating an example of use of the patch.

FIG. 9 is a plan view illustrating a configuration of a patch according to another embodiment.

FIG. 10 is a cross-sectional view illustrating a configuration of the patch.

FIG. 11 is an explanatory view illustrating an example of a configuration and use of a patch according to another embodiment.

FIG. 12 is a bottom view illustrating the configuration of the patch.

FIG. 13 is an explanatory view illustrating an example of a configuration and use of a patch according to another embodiment.

### DETAILED DESCRIPTION

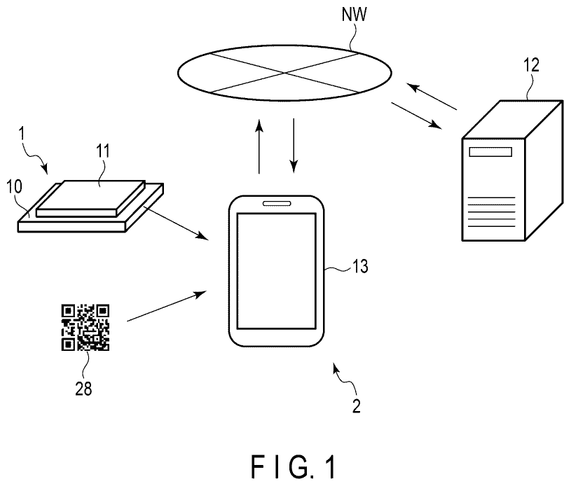

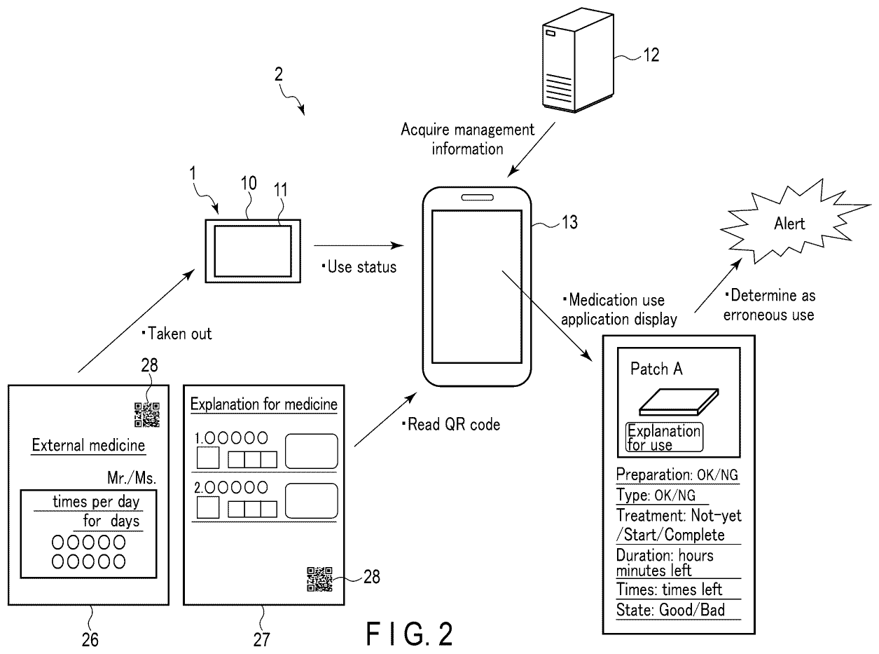

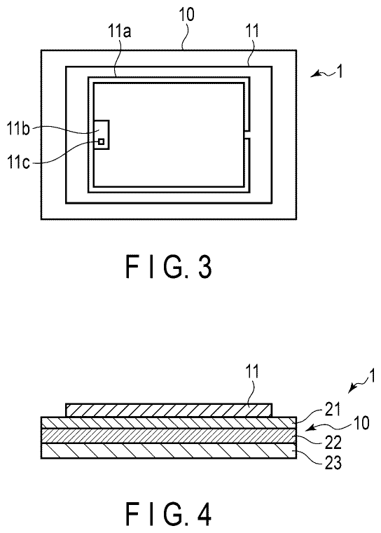

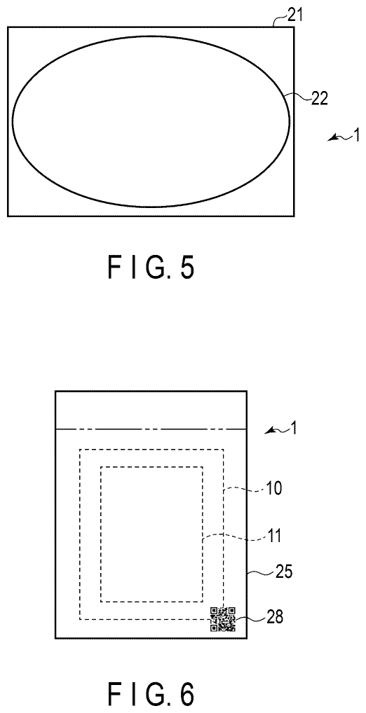

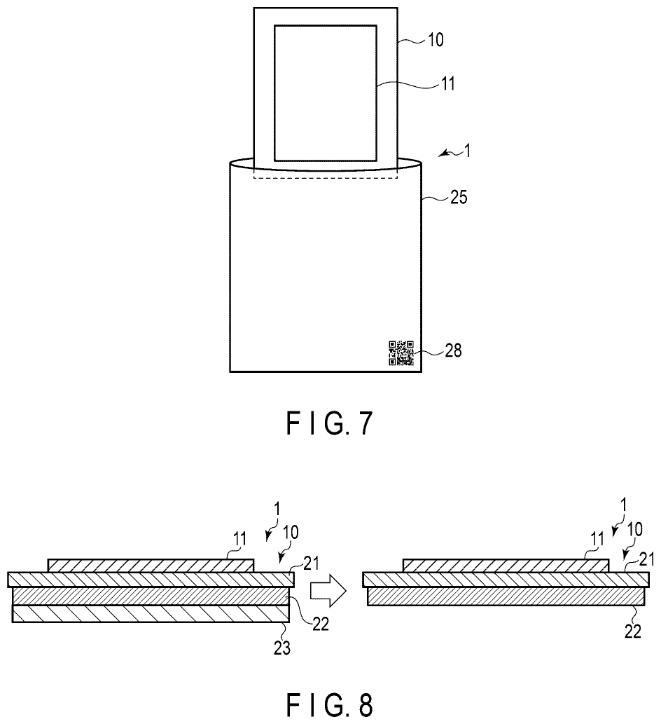

FIG. 1 is an explanatory diagram illustrating a configuration of a medication use support system 2 using a patch 1 according to an embodiment of the present invention, and FIG. 2 is an explanatory diagram illustrating the configuration of the medication use support system 2, and illustrating an example of use. FIG. 3 is a plan view illustrating the configuration of the patch 1, FIG. 4 is a cross-sectional view illustrating the configuration of the patch 1, and FIG. 5 is a bottom view illustrating the configuration of the patch 1. FIG. 6 is a plan view illustrating a state in which the patch 1 is placed in a package 25, and FIG. 7 is an explanatory view illustrating a state in which the package 25 is opened. FIG. 8 is an explanatory view illustrating an example of use of the patch 1.

In the patch 1, for example, a communication device 11 is provided in a patch body 10 which is a medicine, and is formed to be able to wirelessly communicate with a communication terminal. As illustrated in FIGS. 1 and 2, the patch 1 is used, for example, in the medication use support system 2 that performs support (management) when a patient uses the patch 1. First, the medication use support system 2 will be described.

As illustrated in FIGS. 1 and 2, the medication use support system 2 includes the patch 1, a management server 12, and a terminal 13. As illustrated in FIG. 2, the medication use support system 2 allows the terminal 13 to read a code 28, for example, a QR code (registered trademark), as first trigger information, stores the administration method of the patch body 10 determined by a medical institution in the terminal 13, and further uses this as a trigger to acquire management information of the patch body 10, such as a type, description, image, or the like of the patch body 10 from the management server 12 and to store the acquired management information in the terminal 13. In addition, the medication use support system 2 is a system that acquires second trigger information from the communication device 11, acquires and/or determines use status of the patch 1 by the terminal 13 based on the acquired various types of information, and manages the use of medicine by a user (patient). In addition, for example, in a case where the terminal 13 acquires the second trigger information, the medication use support system 2 displays a screen of an application that manages the medication use support on the terminal 13 based on the stored management information, displays the management information of the patch body 10 on the screen of the application, and provides the management information of the patch body 10 to the patient to allow the patient to visually recognize the management information. In addition, the medication use support system 2 determines the use status of the patch body 10 of the patient by the terminal 13 based on the use status acquired from the communication device 11 through communication with the communication device 11, a QR code 28, and the management information acquired from the management server 12, and in a case where erroneous use is determined, the medication use support system 2 issues an alert to the patient by the terminal 13.

As illustrated in FIG. 1, in the medication use support system 2, each component transmits and receives information by communication means. As a specific example, it is assumed that the communication device 11 and the terminal 13 are connected by a relatively short-distance wireless communication means such as Bluetooth (registered trademark). Furthermore, as a specific example, the management server 12 and the terminal 13 are connected via a mobile communication network such as 4G or 5G or a wireless communication line of a relatively long distance such as Wimax via a network NW. Note that the management server 12 and the terminal 13 may be connected via a wired communication line such as an USB (universal serial bus) or a LAN (local area network) connection by a cable. Furthermore, the communication means is not limited thereto, and the communication device 11, the management server 12, and the terminal 13 may be connected by the other communication means than the above-described communication means.

Next, the configuration of the patch 1 will be described. As illustrated in FIG. 3 and FIG. 4, the patch 1 includes the patch body 10 and the communication device 11 detachably provided on the patch body 10.

As shown in FIG. 6, the patch 1 includes, for example, the package 25, and the patch body 10 and the communication device 11 are placed in the package 25 and sealed. For example, when the patch 1 is prescribed by a pharmacy, a predetermined number of the patches 1 packaged in the packages 25 are placed in a medicine bag 26 for management together with an instruction sheet 27, as illustrated in FIG. 2. As shown in FIG. 7, the patch 1 is used, at the time of use, in a state where the package 25 is opened and the patch 1 is taken out from the package 25.

The patch body 10 is used for various diseases and is a patch to be applied to a human body. The patch body 10 is used, for example, in the treatment of bronchial asthma, ischemic heart disease, hypertension, overactive bladder, Alzheimer's disease, Parkinson's disease, attention deficit/hyperactivity disorder, schizophrenia, depression, postmenopausal osteoporosis, nausea, allergy, analgesia, smoking cessation, infertility, and the like. The patch body 10 supplies the medicine into the body of the patient by transdermal absorption. The type, time, the number of times, and the like of use of the patch body 10 are determined.

As a specific example, as shown in FIG. 3 and FIG. 4, the patch body 10 includes a support body 21 having a sheet-like shape, a plaster 22 provided on one main surface of the support body 21, and a protective film 23 that protects the surface of the plaster 22. The patch body 10 is, for example, Estrana tape (manufactured by HISAMITSU Pharmaceutical Co., INC.).

The plaster 22 is provided on one main surface of the support body 21. In addition, the communication device 11 is provided on another main surface of the support body 21. That is, in the patch body 10, the communication device 11 is provided on the surface of the support body 21 opposite to the surface provided with the plaster 22 that comes into contact with the patient's skin. The support body 21 is formed of a material having adhesiveness, or an adhesive layer having adhesiveness is formed on the surface on which the communication device 11 is provided, and the communication device 11 is detachably attached to the support body 21. For example, the support body 21 to which the communication device 11 is detachably attached may be provided with an adhesive on the surface on which the communication device 11 is provided, or the entire support body 21 may be formed of a material having adhesiveness. As a specific example, the support body 21 is formed by an adhesive pad or includes a layer containing an adhesive polymer. Further, the support body 21 may be formed of a material having a function of shielding an electromagnetic wave.

In the support body 21, the plaster 22 is provided on one main surface, and the communication device 11 is provided on another main surface. The support body 21 has a main surface formed in the same shape as the plaster 22 and the communication device 11 or in a shape larger than the plaster 22 and the communication device 11. As a specific example, the main surface of the support body 21 is formed in a rectangular shape larger than the communication device 11 and the plaster 22. The support body 21 is formed in a rectangular shape.

The plaster 22 is formed of a medicine and an adhesive. The plaster 22 is configured such that the contained medicine can be percutaneously absorbed by contacting the skin. The shape of the plaster 22 is formed to be the same as or slightly smaller than the size of the support body 21 and larger than the communication device 11 in the main surface direction. As shown in FIG. 5, the plaster 22 is formed in, for example, an elliptical shape. As shown in FIG. 5, the length of the long axis and the length of the single axis of the elliptical shape of the plaster 22 are set to be the same as or slightly smaller than the long side and the short side of the support body 21, and is formed to be larger than the communication device 11.

The protective film 23 protects the plaster 22 by covering the plaster 22 before the plaster 22 is attached to the skin of the patient. When the plaster 22 is attached to the skin of the patient, the protective film 23 is peeled off from the plaster 22 as shown in FIG. 8. The protective film 23 is formed in a shape covering at least the plaster 22. Therefore, the protective film 23 is formed in a shape larger than the shape of the communication device 11, for example. Furthermore, the protective film 23 faces, for example, the communication device 11 provided on the support body 21 in the thickness direction. Here, the thickness direction is, for example, the same direction as the stacking direction of the communication device 11, the support body 21, and the plaster 22.

The protective film 23 is, for example, an electromagnetic wave shielding member formed of a material having a function of shielding an electromagnetic wave. For example, the protective film 23 has a shielding property of shielding an electromagnetic wave. While the protective film 23 protects the plaster 22, the protective film 23 shields the electromagnetic wave from the communication device 11 provided on the support body 21 or allows the communication device 11 to be in an uncommunicable state. For example, while the protective film 23 is attached to the plaster 22, the communication device 11 is brought into an uncommunicable state, and when the protective film 23 is peeled off from the plaster 22, the communication device 11 is activated and the communication device 11 is brought into a communicable state. Such the protective film 23 is formed of, for example, an aluminum foil, a vapor deposited metal film, a hydrogel film, or the like.

The package 25 packages and seals a single patch body 10 or a plurality of patch bodies 10. The package 25 is an electromagnetic wave shielding member formed of a material having a function of shielding an electromagnetic wave. The package 25 has, for example, a shielding property of shielding an electromagnetic wave. While the patch body 10 is packaged by the package 25, the package 25 shields the electromagnetic wave from the communication device 11 provided on the support body 21 or allows the communication device 11 to be in an uncommunicable state.

As illustrated in FIG. 2, the medicine bag 26 is a bag which indicates information such as a name of a patient, usage, dosage, a dispensing date, a name of a pharmacist who dispenses a medicine, a name and a location of a pharmacy, a hospital, a clinic or the like that has dispensed a medicine. As illustrated in FIG. 2, the instruction sheet 27 is a material for patients which indicates medication information such as a name of a medicine, efficacy, usage, dosage, and treatment schedule.

In addition, the code 28 associating information with the patch body 10 is provided by being printed or being attached as a seal on which the code 28 is printed to at least any of the patch body 10, the communication device 11 provided on the patch body 10, the package 25 packaging the patch body 10, the medicine bag 26, and the instruction sheet 27.

The code 28 is, for example, a one-dimensional code or a two-dimensional code, and is a QR code as a specific example. Hereinafter, the code 28 will be described as the QR code 28. The QR code 28 is first trigger information for acquiring information on the patch body 10 to be managed in the terminal 13. The QR code 28 is coded information of the patch body 10. Here, the information on the patch body 10 can be, for example, information on the patch body 10 itself, information on an administration method or the like determined in advance for the patch body 10, or information on the use of a medicine for each patient by a doctor or a pharmacist, such as a method of using the patch body 10 according to the patient (usage, dosage, treatment schedule). Note that the first trigger information only needs to be associated with the information acquired by the terminal 13 from the management server 12, and thus may be patient information instead of the information of the patch body 10. That is, the first trigger information may include other information in addition to the information of the patch body 10, or may be the information of the patch body 10 itself.

The communication device 11 performs wireless communication with the terminal 13. The radio wave system of the communication device 11 is general, and any frequency band of 433 MHz, 900 MHz band, or 2.45 GHz may be used regardless of the format as long as the terminal 13 can read the recorded information. The wireless communication technology used for the communication device 11 is, for example, Bluetooth (registered trademark) or Bluetooth Low Energy (BLE) (registered trademark). The communication device 11 may be, for example, an active tag or a passive tag.

In a case where the communication device 11 is an active tag, the communication device 11 may have a configuration including a battery, a power supply circuit, and the like, or may be a tag that performs energy harvesting. Preferably, the communication device 11 performs energy harvesting and is a battery-less tag. That is, the communication device 11 is a radio tag that operates by obtaining energy from surrounding electromagnetic waves. For example, examples of an energy harvesting type radio tag include an IoT sensing label “Wiliot IoT pixel” (manufactured by Wiliot). The communication device 11 may also be a type in which a temperature sensor is built.

The communication device 11 is formed in a rectangular shape or a substantially rectangular shape. As a specific example, the communication device 11 includes, for example, a tag antenna 11a including a matching circuit and an IC chip 11b. Furthermore, the communication device 11 includes, for example, a temperature sensor 11c. The IC chip 11b stores unique tag identification information, and transmits the unique tag identification information at the time of communication with the terminal 13. The temperature sensor 11c is built in the IC chip 11b, for example. The communication device 11 may be an RFID tag that communicates with the terminal 13 via a reader-writer.

The communication device 11 is fixed to the support body 21 of the patch body 10, for example, and is packaged in the package 25 together with the patch body 10. The communication device 11 may be attached by a patient to the support body 21 of the patch body 10 taken out from the package 25.

When the plaster 22 is protected by the protective film 23 and when the communication device 11 is packaged in the package 25, the communication device 11 is shielded from the electromagnetic wave and is to be uncommunicable. Note that the communication device 11 may be configured to be uncommunicable when the patch body 10 is attached to the skin of the patient. When the communication device 11 is taken out from the package 25 and the protective film 23 is peeled off before being attached to the skin, the shielding of the electromagnetic wave is released and is to be communicable. The communication device 11 transmits the second trigger information to the terminal 13 by communicating with the terminal 13. In addition to the second trigger information, the communication device 11 may transmit information of the patch body 10 on which the communication device 11 is provided to the terminal 13.

In the patch 1 configured as described above, the protective film 23 covering the plaster 22 is configured to have a function of shielding the electromagnetic wave, so that the communication device 11 can be brought into an uncommunicable state when the protective film 23 covers the plaster 22 before the patch body 10 is used. In addition, when the patch body 10 is used, the protective film 23 is peeled off from the plaster 22, so that the communication device 11 can be activated to be in the communicable state.

As described above, in the patch 1, since the communication device 11 can be brought into an uncommunicable state before use, and the communication device 11 can be brought into a communicable state only by peeling off the protective film 23, the patient can easily switch the communication state of the communication device 11.

In the patch 1, the communication device 11 can be repeatedly used by detachably adhering the communication device 11 to the support body 21. Therefore, the manufacturing cost of the patch 1 can be suppressed by reusing the communication device 11. In addition, for example, if the patient uses the communication device 11 by attaching and detaching relative to the support body, the communication device 11 may be provided in one of the plurality of prescribed patches 1, and the communication device 11 may not be provided in the other patches 1, that is, only the patch body 10 may be prescribed as the other patches 1.

As described above, according to the patch 1 according to the embodiment of the present invention, it is possible to set the uncommunicable state before use and to easily set the communicable state.

Note that the present invention is not limited to the above-described embodiments. In the above-described example, as an example of the patch 1, the configuration in which the patch body 10 includes the protective film 23 that shields electromagnetic waves has been described, but the present invention is not limited thereto.

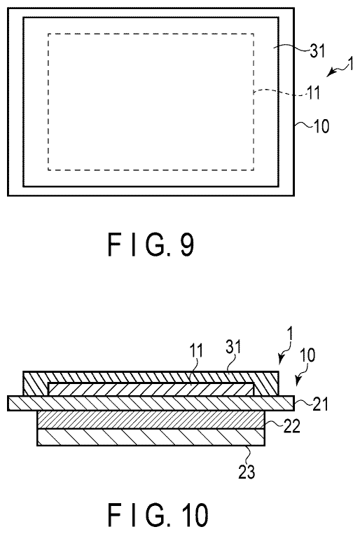

For example, as in another embodiment illustrated in FIG. 9 and FIG. 10, the patch 1 may include a second protective film 31 as an electromagnetic wave shielding member that covers the communication device 11 provided on the support body 21. The second protective film 31 is formed in a shape larger than that of the communication device 11. The second protective film 31 is, for example, an electromagnetic wave shielding member formed of a material having a function of shielding an electromagnetic wave.

For example, the second protective film 31 has a shielding property of shielding electromagnetic waves. The second protective film 31 covers the communication device 11 and protects the communication device 11. While the second protective film 31 protects the communication device 11, the second protective film 31 shields the electromagnetic wave from the communication device 11 or allows the communication device 11 to be in an uncommunicable state. For example, when the second protective film 31 is attached to the communication device 11, the communication device 11 is brought into an uncommunicable state, and when the second protective film 31 is peeled off from the communication device 11, the communication device 11 is activated and the communication device 11 is brought into a communicable state. Such a second protective film 31 is formed of, for example, an aluminum foil, a vapor deposited metal film, a hydrogel film, or the like.

As described above, in a case where the patch 1 has the second protective film 31, the first protective film (protective film) 23 that protects the plaster 22 may have a shielding property to shield the electromagnetic wave, and may not have a shielding property to shield the electromagnetic wave. That is, the patch 1 may be configured to include at least one of the first protective film 23 and the second protective film 31, and at least one of the first protective film 23 and the second protective film 31 may have a shielding property to shield the electromagnetic wave. Therefore, as another example, a configuration may be employed in which the first protective film 23 and the second protective film 31 are provided, the first protective film 23 may be formed as an electromagnetic wave shielding member that shields an electromagnetic wave, and the second protective film 31 may not have a shielding property that shields an electromagnetic wave.

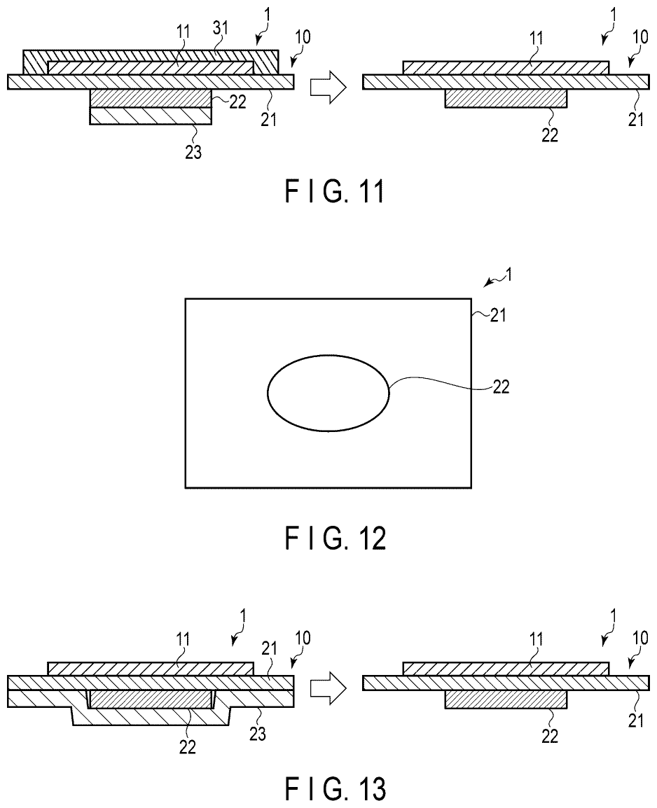

Furthermore, in the above-described example, it is described that the plaster 22 is the same as or slightly smaller than the support body 21 and is larger than the communication device 11, that is, the length of the long axis and the length of the single axis of the elliptical shape of the plaster 22 are set to be the same as or slightly smaller than the long side and the short side of the support body 21, and is formed to be larger than the communication device 11, as a specific example. However, the shape of the plaster 22 is not limited thereto. For example, as in the patch 1 of another embodiment illustrated in FIG. 11 and FIG. 12, the plaster 22 may be configured to be considerably smaller than the support body 21 and smaller than the communication device 11.

As a specific example, the plaster 22 may be formed in an elliptical shape, and the length of the long axis and the length of the single axis of the elliptical shape may be half or less of the long side and the short side of the support body 21, and may be formed smaller than the communication device 11. In the patch 1 having such a configuration, since the protective film 23 having the same shape as the plaster 22 is smaller than the communication device 11, it is difficult to shield the electromagnetic wave, and the second protective film 31 having a shielding property for shielding the electromagnetic wave may be provided.

As another example, as shown in FIG. 13, the shape of the protective film 23 may be larger than the plaster 22 and the communication device 11. Note that, in a case where the protective film 23 is configured to be larger than the plaster 22, when a gap is generated between the protective film 23 and the support body 21, the communication device 11 may be in a communicable state. Therefore, it is preferable that the entire circumference of the outer peripheral edge of the protective film 23 is in contact with at least the support body 21.

Further, in the above-described example, the configuration in which the patch 1 includes the package 25 that shields the electromagnetic wave has been described, but the present invention is not limited thereto. The patch 1 may be configured to include the package 25 that does not shield the electromagnetic wave, or may be configured not to include the package 25.

That is, the present invention is not limited to the above embodiment, and various modifications can be made in the implementation stage without departing from the gist thereof. In addition, each embodiment may be implemented in a suitable combination with others. If this is the case, combined effects can be attained. Furthermore, the above embodiments include inventions at various stages, and various inventions can be extracted by an appropriate combination of a plurality of disclosed constitutional requirements. For example, even if some constituent elements are deleted from all the constituent elements shown in the embodiments, when the problem can be solved and the effect can be obtained, the configuration from which the constituent elements are deleted can be extracted as the invention.

1. A patch comprising:
a support body;
a plaster provided on one main surface of the support body;
a communication device provided on another main surface of the support body and wirelessly communicating with a communication terminal; and
an electromagnetic wave shielding member provided on at least one of the plaster and the communication device and allowing the communication device to be in an uncommunicable state.

2. The patch according to claim 1, wherein the electromagnetic wave shielding member is a protective film covering the plaster.

3. The patch according to claim 2, wherein the plaster is larger than the communication device.

4. The patch according to claim 1, wherein
the plaster is smaller than the communication device, and
the electromagnetic wave shielding member is a protective film that covers the plaster and is larger than the communication device.

5. The patch according to claim 1, comprising a first protective film covering the plaster,
wherein the electromagnetic wave shielding member is a second protective film covering the communication device.

6. The patch according to claim 1, wherein the support body has adhesiveness with which the communication device is detachably attached.

7. The patch according to claim 1, comprising a package that shields an electromagnetic wave,
wherein the support body, the plaster, the communication device, and the electromagnetic wave shielding member are placed in the package.

8. The patch according to claim 1, wherein the communication device is a radio tag that operates by obtaining energy from a surrounding electromagnetic wave.

9. The patch according to claim 2, wherein the communication device is a radio tag that operates by obtaining energy from a surrounding electromagnetic wave.

10. The patch according to claim 3, wherein the communication device is a radio tag that operates by obtaining energy from a surrounding electromagnetic wave.

11. The patch according to claim 4, wherein the communication device is a radio tag that operates by obtaining energy from a surrounding electromagnetic wave.

12. The patch according to claim 5, wherein the communication device is a radio tag that operates by obtaining energy from a surrounding electromagnetic wave.

13. The patch according to claim 6, wherein the communication device is a radio tag that operates by obtaining energy from a surrounding electromagnetic wave.

14. The patch according to claim 8, wherein the communication device is a radio tag that operates by obtaining energy from a surrounding electromagnetic wave.
